(19) United States
(12) Patent Application Publication
(10) Pub. No.: US 2021/0401569 A1
(43) Pub. Date: Dec. 30, 2021

(54) LIGAMENT FIXATION DEVICE, LIGAMENT FIXATION SYSTEM, AND LIGAMENT FIXATION METHOD

(71) Applicant: OLYMPUS CORPORATION, Tokyo (JP)

(72) Inventor: Masaki HAYASHI, Tokyo (JP)

(73) Assignee: OLYMPUS CORPORATION, Tokyo (JP)

(21) Appl. No.: 17/470,832

(22) Filed: Sep. 9, 2021

Related U.S. Application Data
(63) Continuation of application No. PCT/JP2019/009822, filed on Mar. 11, 2019.

(51) Int. Cl. A61F 2/08

(52) U.S. Cl. CPC A61F 2/0811; A61F 2220/0016; A61F 2002/0882

(57) ABSTRACT

A ligament fixation device for fixing a ligament to a bone tunnel includes: a fixation device body having a flat surface, wherein the fixation device body has a mounting section for mounting the ligament or a pulling member thereto and a torque application portion configured to apply torque about an axis orthogonal to the flat surface, the pulling member being provided for mounting the ligament, and wherein the mounting section includes a pair of through-holes provided in the noncircular flat surface.

### CROSS-REFERENCE TO RELATED APPLICATIONS

This is a continuation of International Application PCT/JP2019/009822 which is hereby incorporated by reference herein in its entirety.

### TECHNICAL FIELD

The present disclosure relates to ligament fixation devices, ligament fixation systems, and ligament fixation methods.

### BACKGROUND ART

A known fixation device is used for fixing a suture to a bone (e.g., see Patent Literature 1).

This fixation device is provided with an elongated attachment section for attaching the suture to a central area in the longitudinal direction. After the fixation device is inserted into a bone tunnel formed in the bone to a depth where the cancellous bone exists, the suture attached to the attachment section is pulled so that the fixation device is rotated around an axis intersecting the longitudinal axis of the bone tunnel. Accordingly, the opposite ends of the fixation device become hooked to the cortical bone, whereby the fixation device is prevented from falling out of the bone tunnel.

### CITATION LIST

### Patent Literature

{PTL 1}

U.S. Pat. No. 523,787

### SUMMARY OF INVENTION

An aspect of the present disclosure provides a ligament fixation device for fixing a ligament to a bone tunnel and including a fixation device body having a noncircular flat surface. The fixation device body has a mounting section for mounting the ligament or a pulling member thereto and a torque applier capable of applying torque around an axis orthogonal to the flat surface. The pulling member is provided for mounting the ligament. The mounting section includes at least two through-holes provided in the noncircular flat surface.

### BRIEF DESCRIPTION OF DRAWINGS

FIG. 1 is a perspective view illustrating a ligament fixation system according to an embodiment of the present disclosure.

FIG. 2 is a front view illustrating a state where a ligament fixation device constituting the ligament fixation system in FIG. 1 is aligned with a cross section of a bone tunnel.

FIG. 3 is a front view illustrating a state where the ligament fixation device in FIG. 2 is rotated relative to the bone tunnel.

FIG. 4 is a partially-cutaway side view illustrating a state where a reconstruction ligament is mounted to the ligament fixation system in FIG. 1.

FIG. 5 is a partial perspective view illustrating an anterior cruciate ligament originally existing in a knee joint.

FIG. 6 is a vertical sectional view illustrating the location and angle of a thigh bone tunnel formed in a thigh bone.

FIG. 7 is a vertical sectional view illustrating a state where the ligament fixation device of the ligament fixation system in FIG. 4 is inserted into the thigh bone tunnel.

FIG. 8 is a vertical sectional view illustrating a state where the ligament fixation device is rotated within the thigh bone tunnel from the state in FIG. 7.

FIG. 9 is a vertical sectional view illustrating a state where an installation jig is removed from the thigh bone tunnel from the state in FIG. 8.

FIG. 10 is a cross-sectional view illustrating spaces formed as a result of rotating the ligament fixation device inserted in the thigh bone tunnel from the state in FIG. 2 to the state in FIG. 3.

FIG. 11 is a front view illustrating a modification of the ligament fixation device in FIG. 2.

FIG. 12 is a front view illustrating another modification of the ligament fixation device in FIG. 2.

FIG. 13 is a perspective view illustrating another modification of the ligament fixation device in FIG. 2.

FIG. 14 is a front view illustrating a modification of a mounting section of the ligament fixation device in FIG. 2.

FIG. 15 is a front view illustrating another modification of the mounting section of the ligament fixation device in FIG. 2.

FIG. 16 is a front view illustrating another modification of the mounting section of the ligament fixation device in FIG. 2.

FIG. 17 is a front view illustrating a modification of a torque applier of the ligament fixation device in FIG. 2.

FIG. 18 is a perspective view illustrating the relationship between a rod-like ligament fixation device and the installation jig.

FIG. 19 is a perspective view illustrating a positional-displacement prevention member to be additionally mounted to the ligament fixation device in FIG. 2.

FIG. 20 is a perspective view illustrating a state where the positional-displacement prevention member in FIG. 19 is mounted to the ligament fixation device in FIG. 2 within the bone tunnel.

FIG. 21 is a perspective view of the ligament fixation device and illustrates a modification of the positional-displacement prevention member.

FIG. 22 is a perspective view of the ligament fixation device and illustrates another modification of the positional-displacement prevention member.

FIG. 23 is a perspective view of the ligament fixation device and illustrates another modification of the positional-displacement prevention member.

FIG. 24 is a vertical sectional view illustrating a state where the reconstruction ligament is fixed within the bone tunnel by using the ligament fixation device having the positional-displacement prevention member in FIG. 23.

FIG. 25 is a front view illustrating another modification of the ligament fixation device in FIG. 2.

FIG. 26 is a vertical sectional view illustrating a state where the reconstruction ligament is hooked to the ligament fixation device in FIG. 25.

FIG. 27 is a flowchart illustrating a ligament fixation method according to an embodiment of the present disclosure.

### DESCRIPTION OF EMBODIMENTS

A ligament fixation device 1, a ligament fixation system 100, and a ligament fixation method according to an embodiment of the present disclosure will be described below with reference to the drawings.

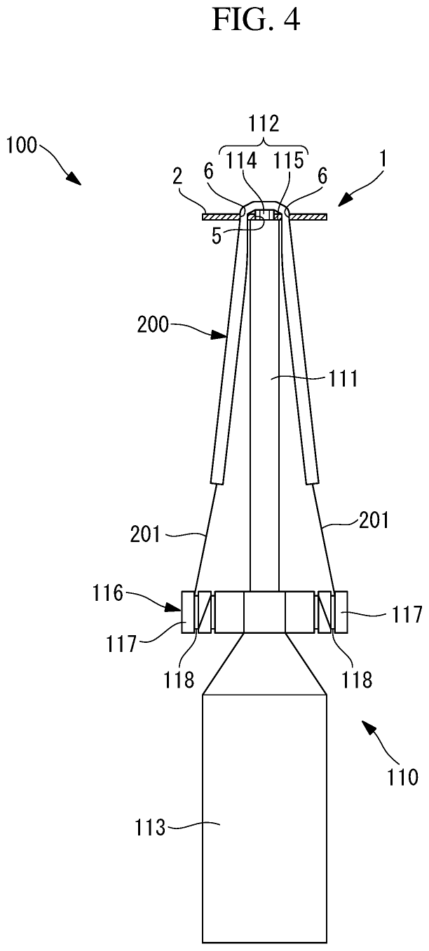

When mending the anterior cruciate ligament or the posterior cruciate ligament of a knee joint damaged as a result of excessive stress applied thereto during a sports activity, for example, a tendon autograft is extracted as a ligament graft and is used as a reconstruction ligament (i.e., ligament, see FIG. 4) 200. Alternatively, an artificial ligament that has been artificially fabricated may be used. Normally, the reconstruction ligament 200 used is obtained by connecting artificial ligaments or high-strength sutures as tension threads (pulling members) 201 to the opposite ends of a ligament graft.

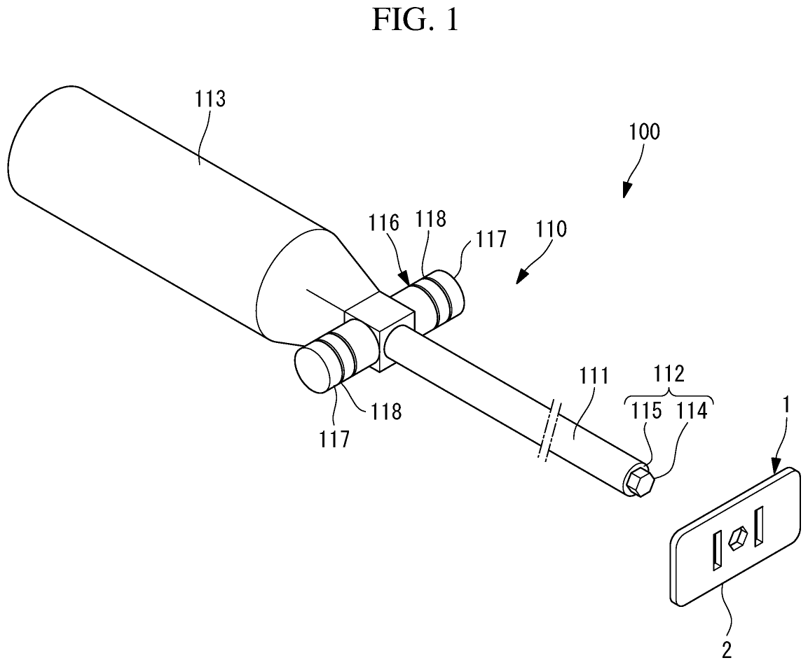

As shown in FIG. 1, the ligament fixation system 100 according to this embodiment includes the ligament fixation device 1 and an installation jig 110.

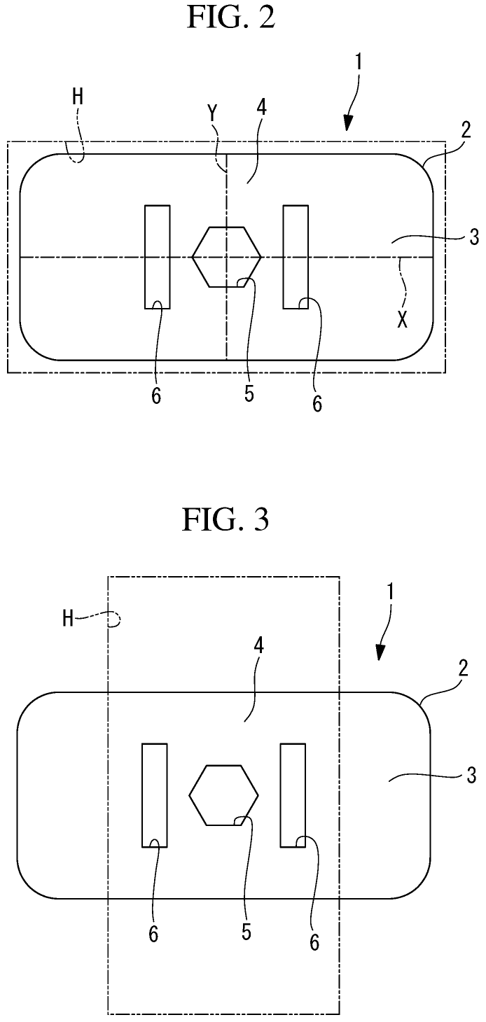

As shown in FIG. 2, the ligament fixation device 1 according to this embodiment includes a tabular fixation device body 2 having a rectangular shape with a longitudinal axis (i.e., first axis) X and a lateral axis (i.e., second axis) Y. The fixation device body 2 is composed of a material having high biocompatibility and sufficient strength.

The biocompatible material used in the fixation device body 2 is preferably a metallic material that can ensure sufficient strength and resiliency over a long period of time, and may be freely selected from, for example, stainless steel (such as SUS316L), pure titanium, and a titanium alloy. Alternatively, the biocompatible material used in the fixation device body 2 may be a bioabsorbable material, such as polylactate (PLLA), polyglycolide (PGA), or a magnesium alloy, or may be a highly functional resin, such as polyether ether ketone.

The fixation device body 2 includes a wide section 3 in the direction of the longitudinal axis X, and also includes a narrow section 4 having a width smaller than the wide section 3 in the direction of the lateral axis Y that is orthogonal to the longitudinal axis X.

The fixation device body 2 is provided with a first through-hole (i.e., through-hole, torque applier) 5 and second through-holes (i.e., through-hole, mounting section) 6 extending therethrough in the thickness direction. The first through-hole 5 is provided at the center of the fixation device body 2 and has a regular hexagonal cross-sectional shape.

The second through-holes 6 are where the reconstruction ligament 200 is to be mounted and are two slits disposed at opposite sides of the first through-hole 5 in the direction of the longitudinal axis X and extending parallel to each other along the lateral axis Y. In the direction of the longitudinal axis X, the second through-holes 6 are disposed within a minimum tunnel diameter of a bone tunnel H to be formed in a bone. As shown in FIG. 3, the second through-holes 6 may be disposed at intermediate locations, in the direction of the longitudinal axis X, within the minimum tunnel diameter of the bone tunnel H to be formed in the bone.

As shown in FIG. 1, the installation jig 110 includes a rod-like shaft 111, a connector 112 provided at one end of the shaft 111, and a handle (i.e., torque supplier) 113 provided at the other end of the shaft 111.

The shaft 111 has an outer diameter smaller than the narrow section 4 of the fixation device body 2.

The connector 112 includes a regular-hexagonal-prismatic engagement section 114 engageable with the first through-hole 5 provided in the fixation device body 2 in a state where a longitudinal axis Z of the shaft 111 and an axis orthogonal to the surface of the fixation device body 2 are aligned with each other, and also includes a step 115 on which the surface of the fixation device body 2 abuts when the engagement section 114 is engaged with the first through-hole 5.

The handle 113 has a cylindrical shape with an outer diameter sufficiently larger than that of the shaft 111 and can easily supply torque to the shaft 111 by being gripped and twisted around the longitudinal axis Z of the shaft 111 by an operator. Alternatively, the handle 113 may have a shape other than a cylindrical shape, and may be subjected to an embossing treatment or a non-slip treatment.

As shown in FIG. 4, the handle 113 is provided with a thread securing section 116 near the shaft 111. The thread securing section 116 is where the tension threads 201 at the opposite ends of the reconstruction ligament 200 mounted to the ligament fixation device 1 are secured. The thread securing section 116 is provided with a plurality of circumferential grooves 118 in two columnar sections 117 extending orthogonally to the longitudinal axis Z of the shaft 111. The circumferential grooves 118 are provided for winding and securing the tension threads 201 thereto.

The ligament fixation method according to this embodiment using the ligament fixation device 1 and the ligament fixation system 100 having the above-described configuration will be described below.

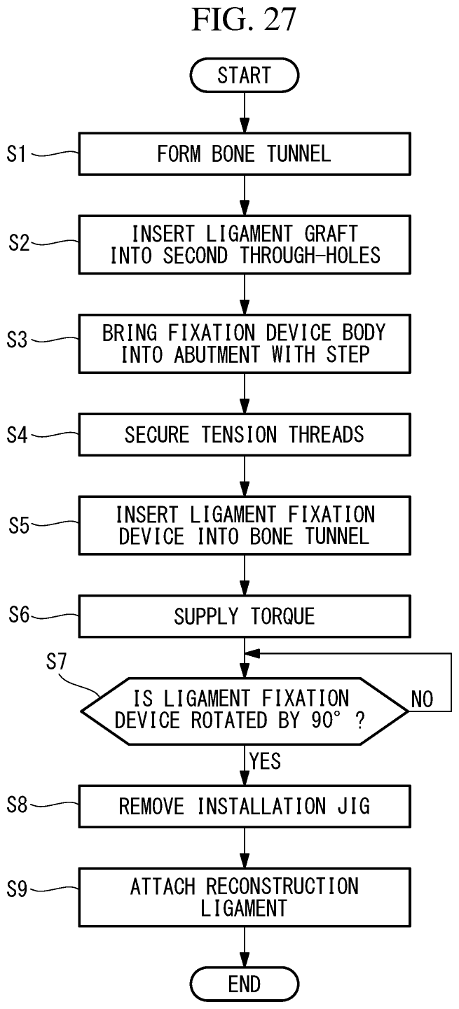

As shown in FIG. 27, the ligament fixation method according to this embodiment involves fixing the reconstruction ligament 200 to a thigh bone (i.e., bone) B for reconstructing the anterior cruciate ligament of a knee joint.

First, a thigh bone tunnel (i.e., bone tunnel) H is formed in the thigh bone B (step S1). The bone tunnel H is formed to a depth where cancellous bone C exists within the thigh bone B.

As shown in FIG. 5, the anterior cruciate ligament originally connects an inner location of a lateral condyle B1 disposed at the shin-bone-D end of the thigh bone B with a substantially central location on the inner front side of an inner joint surface of the shin bone D that receives a medial condyle B2 disposed next to the inner side of the lateral condyle B1.

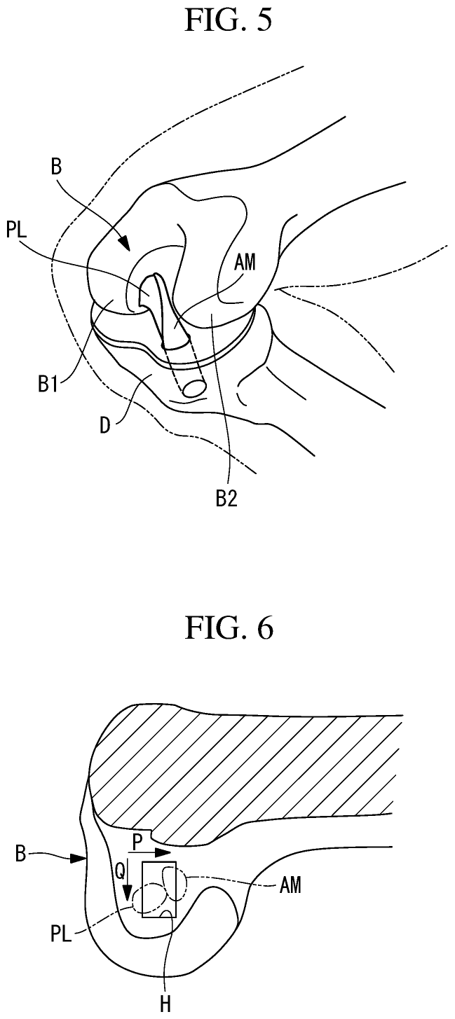

Anatomically, the anterior cruciate ligament is further divided into an antero-medial bundle AM and a postero-medial bundle PL. As indicated by chain lines in FIG. 6, the antero-medial bundle AM and the postero-medial bundle PL are attached substantially next to each other in the longitudinal direction of the thigh bone B at the inner location of the lateral condyle B1.

As indicated by a solid line in FIG. 6, after the damaged anterior cruciate ligament has been removed, the thigh bone tunnel H is formed at the inner location of the lateral condyle B1 originally connected with the anterior cruciate ligament, such as to extend from the surface of the lateral condyle B1 to a depth where the cancellous bone C exists inside.

As indicated by a chain line in FIG. 2, in this embodiment, the thigh bone tunnel H has a fixed rectangular (i.e., noncircular or polygonal) cross-sectional shape that is slightly larger than the outer rectangular shape of the fixation device body 2. Specifically, the maximum length of the ligament fixation device 1 is smaller than the maximum length of the bone tunnel H and larger than the minimum dimension thereof. In other words, the thigh bone tunnel H has a maximum inner dimension that is slightly larger than the width of the wide section 3 of the fixation device body 2, and has a minimum inner dimension that is slightly larger than the width of the narrow section 4 of the fixation device body 2 and sufficiently smaller than the width of the wide section 3. More specifically, the wide section 3 of the ligament fixation device 1 is slightly smaller than the maximum inner dimension of the thigh bone tunnel H and sufficiently larger than the minimum inner dimension thereof. The narrow section 4 of the ligament fixation device 1 is slightly smaller than the minimum inner dimension of the thigh bone tunnel H.

As shown in FIG. 6, the thigh bone tunnel H is formed at an angle such as to have the minimum inner dimension in the lengthwise direction P of the thigh bone B and the maximum inner dimension in a direction Q orthogonal to the lengthwise direction P of the thigh bone B. The thigh bone tunnel H having the rectangular cross-sectional shape can be easily formed by using, for example, an ultrasonic probe described in WO 2018/078826.

As indicated by a dashed line in FIG. 5, by using a known technique, a bone tunnel extending toward the shin bone D is formed in the shin bone D at the substantially central location on the inner front side of the inner joint surface originally connected with the anterior cruciate ligament.

Subsequently, as shown in FIG. 4, the reconstruction ligament 200 is bent in half, the tension threads 201 at the opposite ends are respectively extended and pulled through the two second through-holes 6 of the fixation device body 2 from one face (defined as “front face”) toward the other face (defined as “rear face”), and the ligament graft is inserted through the second through-holes 6 until the segment near the bent location of the ligament graft abuts on the one face of the fixation device body 2 (step S2). Consequently, the reconstruction ligament 200 is mounted to the ligament fixation device 1.

In this state, the front side of the first through-hole 5 is covered by the ligament graft. Then, as shown in FIG. 4, the engagement section 114 of the connector 112 of the installation jig 110 is engaged with the first through-hole 5 from the rear side at which the first through-hole 5 is exposed, and the rear face of the fixation device body 2 is brought into abutment with the step 115 of the connector 112 (step S3). Subsequently, the tension threads 201 at the opposite ends of the reconstruction ligament 200 are wound around and secured to the thread securing section 116 provided in the installation jig 110 (step S4).

Accordingly, as shown in FIG. 4, the tabular ligament fixation device 1 having the first through-hole 5 engaged with the engagement section 114 can be attached to the distal end of the rod-like shaft 111 in such a manner as to prevent the tabular ligament fixation device 1 from falling off while being positioned orthogonally to the longitudinal axis Z of the shaft 111.

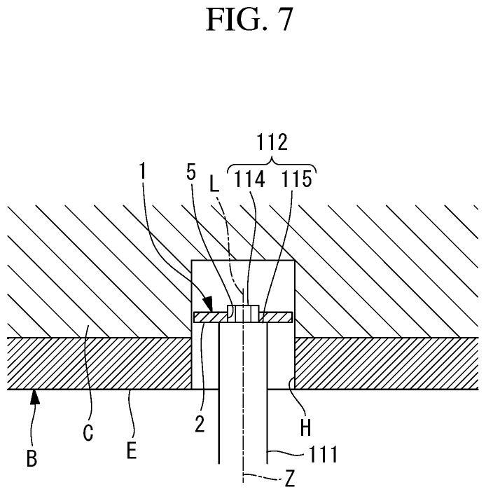

Then, as shown in FIG. 7, the ligament fixation device 1 equipped with the reconstruction ligament 200 attached to the distal end of the shaft 111 of the installation jig 110 is inserted into the thigh bone tunnel H (step S5). In FIG. 7, the reconstruction ligament 200 has been omitted to provide a clear illustration.

In this case, the ligament fixation device 1 is inserted into the thigh bone tunnel H in a state where a longitudinal axis (i.e., axis) L of the thigh bone tunnel H is aligned with the longitudinal axis Z of the shaft 111.

As shown in FIG. 7, the ligament fixation device 1 is inserted into the thigh bone tunnel H while maintaining its position orthogonal to the axis of the thigh bone tunnel H, and is inserted to a depth where the cancellous bone C exists within the thigh bone B. At this point, the operator twists the handle 113 of the installation jig 110 disposed outside the thigh bone B, so that torque acting around the longitudinal axis Z is supplied to the shaft 111 (step S6).

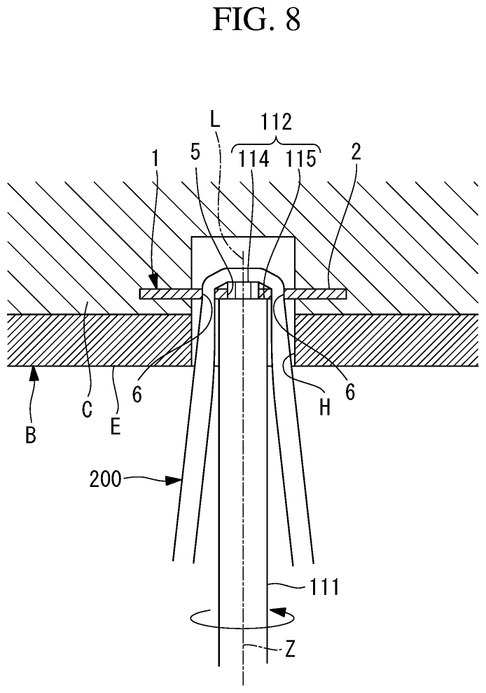

Because the hexagonal engagement section 114 at the distal end of the shaft 111 is engaged with the hexagonal first through-hole 5 of the ligament fixation device 1, the torque acting on the shaft 111 is applied to the ligament fixation device 1, so that the ligament fixation device 1 rotates around the longitudinal axis Z of the shaft 111 within the cancellous bone C. Specifically, the ligament fixation device 1 is rotated around the longitudinal axis L of the bone tunnel H. Then, as shown in FIGS. 3 and 8, when rotated by 90° around the longitudinal axis Z of the shaft 111, the ligament fixation device 1 stops rotating (step S7).

Accordingly, the opposite ends of the wide section 3 of the fixation device body 2 dig into the cancellous bone C located at the outer side of the inner wall of the thigh bone tunnel H in the direction of the minimum inner dimension thereof. Even if the fixation device body 2 receives a pulling force acting radially outward of the thigh bone tunnel H in this state, the ligament fixation device 1 is prevented from falling out of the thigh bone tunnel H since the opposite ends of the wide section 3 are hooked to cortical bone E having higher rigidity than the cancellous bone C.

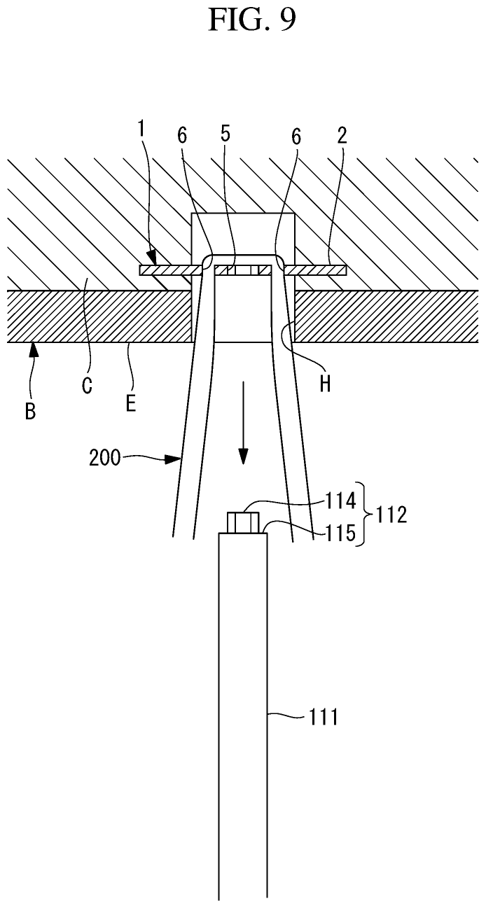

Subsequently, the installation jig 110 is pulled in the axial direction of the thigh bone tunnel H, so that the engagement section 114 of the installation jig 110 engaged with the first through-hole 5 of the fixation device body 2 is pulled out of the first through-hole 5, thereby removing the installation jig 110 from the thigh bone tunnel H (step S8). Accordingly, as shown in FIG. 9, the ligament fixation device 1 equipped with the reconstruction ligament 200 is attached to the thigh bone B, and the reconstruction ligament 200 is attached to the thigh bone B.

In this state, the second through-holes 6 provided in the fixation device body 2 are disposed within the inner wall of the thigh bone tunnel H. Accordingly, the reconstruction ligament 200 mounted to the fixation device body 2 by extending through the two second through-holes 6 extends in the form of two ligaments from the thigh bone tunnel H to the outside of the thigh bone B.

In this state, the two second through-holes 6 are arranged in the longitudinal direction of the thigh bone B. Therefore, the two ligaments extending through the thigh bone tunnel H to the outside of the thigh bone B from the two second through-holes 6 are similar to the original antero-medial bundle AM and postero-medial bundle PL in that the two ligaments are fixed at locations substantially next to each other in the longitudinal direction of the thigh bone B at the inner side of the lateral condyle B1 of the thigh bone B. The two pieces of the reconstruction ligament 200 fixed to the thigh bone B are inserted into the bone tunnel in the shin bone D and are attached and fixed to the shin bone D in a tensioned state (step S9). The two pieces of the reconstruction ligament 200 are fixed to the shin bone D by using, for example, a known technique described in Japanese Unexamined Patent Application, Publication No. 2018-117905. Consequently, the anterior cruciate ligament is reconstructed.

Accordingly, with the ligament fixation device 1, the ligament fixation system 100, and the ligament fixation method according to this embodiment, the ligament 200 can be easily fixed to the thigh bone B by simply rotating the ligament fixation device 1 inserted in the thigh bone tunnel H by 90° around the axis of the thigh bone tunnel H.

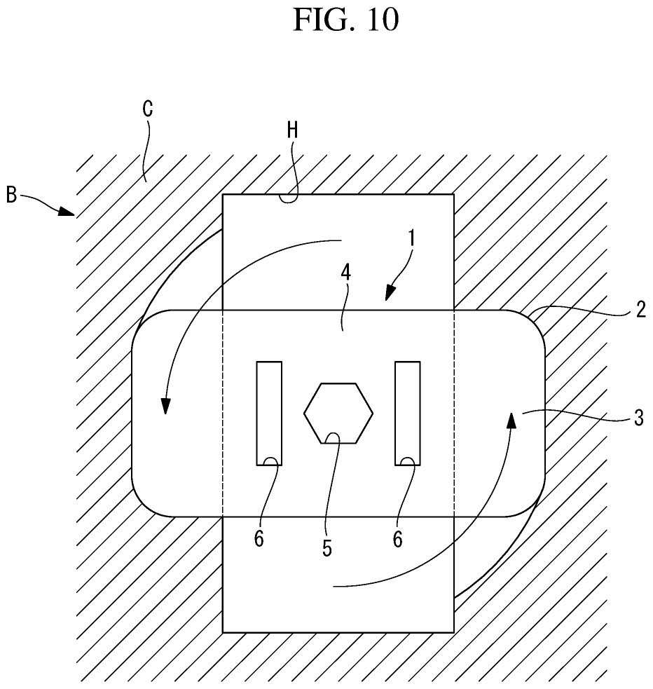

In this case, when the ligament fixation device 1 is rotated within the cancellous bone C, the cancellous bone C disposed at the location where the fixation device body 2 constituting the ligament fixation device 1 passes is removed by the fixation device body 2, so that spaces are formed, as shown in FIG. 10. According to this embodiment, the tabular fixation device body 2 is rotated around the axis orthogonal to the surface thereof in a state where the longitudinal axis Z of the shaft 111 and the axis orthogonal to the surface of the fixation device body 2 are aligned with each other, so that the spaces formed as a result of the removal of the cancellous bone C are disposed adjacent to the fixation device body 2 in the circumferential direction thereof.

Specifically, according to this embodiment, spaces are not formed in areas adjacent to the fixation device body 2 in the thickness direction thereof, so that the fixation device body 2 has no room to move in the axial direction of the thigh bone tunnel H. Thus, even if the reconstructed ligament 200 repeatedly receives tension, the ligament 200 can be maintained in a stable fixed state.

Moreover, because the fixation device body 2 is tabular (broad and flat) and is rotated in a direction parallel to the surface of the fixation device body 2, the amount of the cancellous bone C to be removed as a result of the rotation is equivalent to the thickness of the fixation device body 2 and is thus extremely small. This is advantageous in that the spaces to be formed can be minimized.

Furthermore, the mounting process can be easily performed by simply inserting the ligament graft through the two slit-like second through-holes 6 provided at a distance from each other in the fixation device body 2. Then, in a state where the fixation device body 2 is disposed at the ultimate fixation location by being rotated within the cancellous bone C, the two second through-holes 6 are disposed next to each other in the longitudinal direction of the thigh bone B, so that an arrangement similar to that of the original antero-medial bundle AM and postero-medial bundle PL can be realized. This is advantageous in that the ligament 200 can be reconstructed at an anatomically correct location.

As an alternative to this embodiment in which the fixation device body 2 is described as being rectangular and tabular, the fixation device body 2 used may have any freely-chosen noncircular shape, such as a polygonal shape, an oblong shape, or an elliptical shape, so long as the fixation device body 2 has the wide section 3 and the narrow section 4. Likewise, the cross-sectional shape of the bone tunnel H may be any freely-chosen noncircular shape, such as a polygonal shape, an oblong shape, or an elliptical shape.

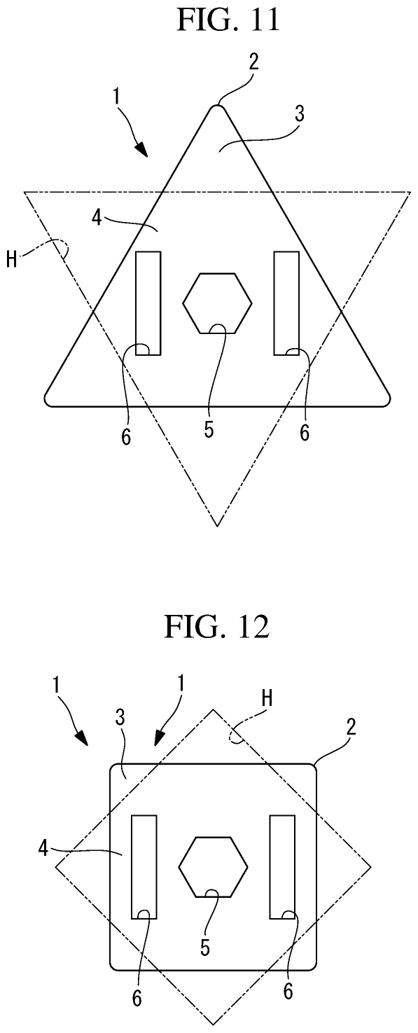

For example, if the fixation device body 2 is triangular, as shown in FIG. 11, the fixation device body 2 can be fixed by being inserted into the bone tunnel H having a complementary cross section and then by being rotated by 60° around the axis of the bone tunnel H. For example, if the fixation device body 2 is square-shaped, as shown in FIG. 12, the fixation device body 2 can be fixed by being inserted into the bone tunnel H having a complementary cross section and then by being rotated by 45° around the axis of the bone tunnel H.

The fixation device body 2 is not limited to being rectangular and may have an ellipsoidal shape or a partially-cutout circular shape. Although the fixation device body 2 is preferably tabular, the fixation device body 2 does not necessarily have to be tabular and may be in the form of a bent plate, a block, or a rod.

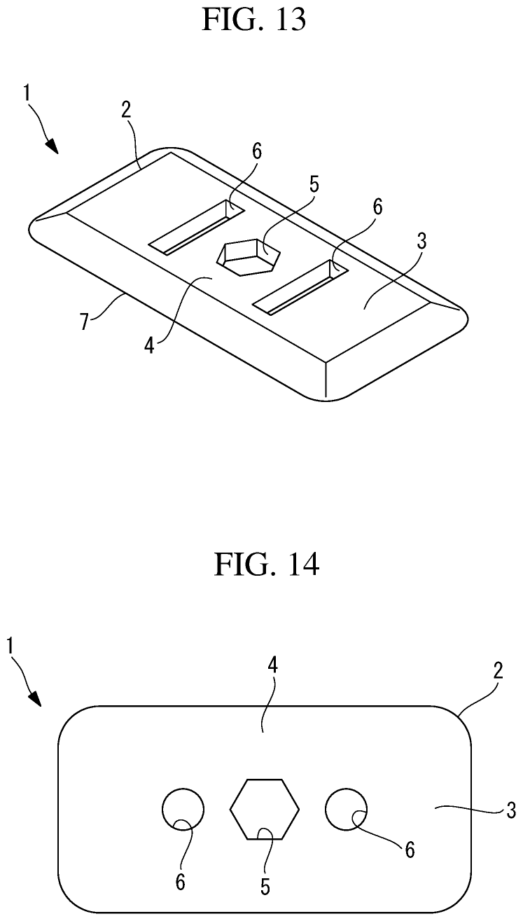

Furthermore, as shown in FIG. 13, the fixation device body 2 may have a sharp edge 7 that extends along the perimeter thereof and that decreases in thickness toward the tip. Accordingly, when the fixation device body 2 is rotated within the cancellous bone C, the sharp edge 7 can easily cut into the cancellous bone C. The sharp edge 7 does not have to be provided along the entire perimeter. For example, if the rotation within the cancellous bone C is to be limited only to one direction, the sharp edge 7 may be provided only in an area serving as the leading edge during the rotation.

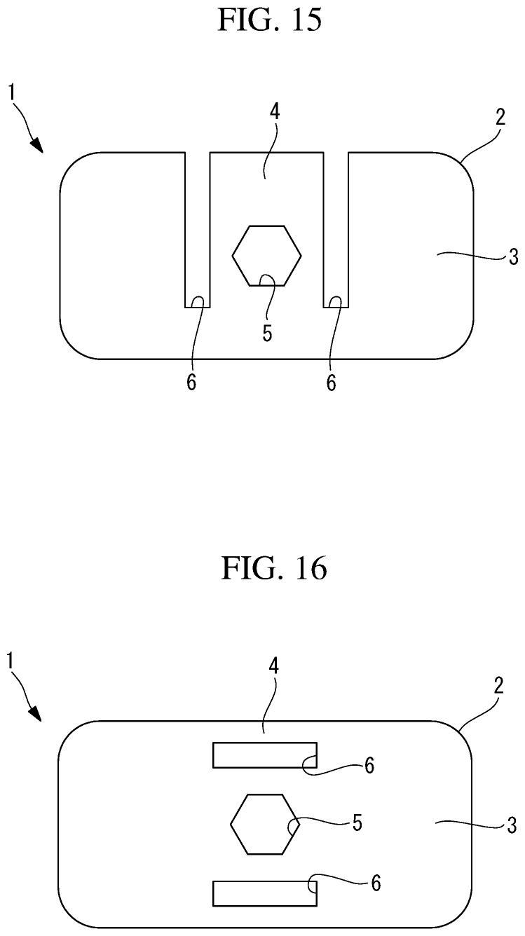

As an alternative to the above example where two slit-like second through-holes 6 are provided as the mounting section, the second through-holes 6 may be circular holes, as shown in FIG. 14, or the second through-holes 6 may be cutouts provided at an edge of the fixation device body 2, as shown in FIG. 15. In this embodiment, the second through-holes 6 are provided at a distance from each other along the longitudinal axis X of the rectangular fixation device body 2. Alternatively, as shown in FIG. 16, the second through-holes 6 may be provided at a distance from each other along the lateral axis Y. In this case, the thigh bone tunnel H may be rotated by 90° around the axis of the thigh bone tunnel H.

As an alternative to the above example where the torque applier used is the hexagonal first through-hole 5, the torque applier may alternatively be a cross-shaped or linear recess or a through-hole connectable with the screwdriver-like connector 112. Specifically, since the torque applier may have any shape so long as the connector 112 provided at the distal end of the shaft 111 of the installation jig 110 can transmit torque around the longitudinal axis Z of the shaft 111, the torque applier may be a single recess or a single through-hole having any freely-chosen noncircular cross-sectional shape. As another alternative, the torque applier may be a screw hole.

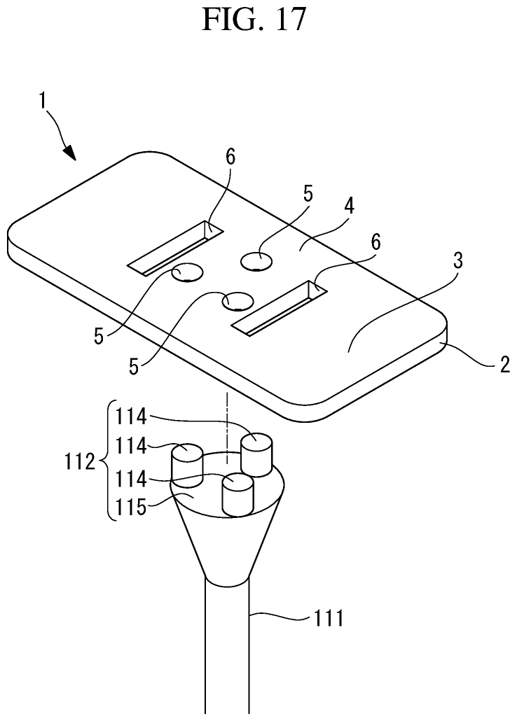

The first through-hole 5 used may be a plurality of through-holes provided at a distance from each other, as shown in FIG. 17. In this case, the distal end of the shaft 111 of the installation jig 110 may be provided with a plurality of columnar engagement sections 114 that simultaneously engage with the plurality of through-holes. In this case, each first through-hole 5 may have any freely-chosen cross-sectional shape, and may be circular. Moreover, the number of through-holes in this case is not limited.

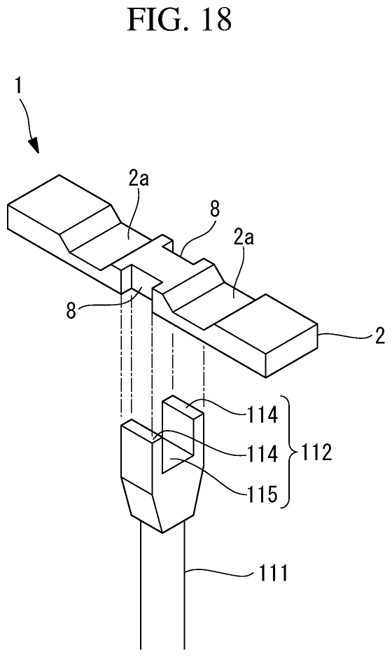

If the fixation device body 2 is rod-like, the torque applier may be two parallel flat surfaces 8 provided at the center of the fixation device body 2, as shown in FIG. 18. In this case, the connector 112 of the installation jig 110 may have a bifurcated structure disposed where the connector 112 clamps the fixation device body 2 by means of the two flat surfaces 8 of the fixation device body 2. In this case, recesses 2a provided in the fixation device body 2 serve as the mounting section.

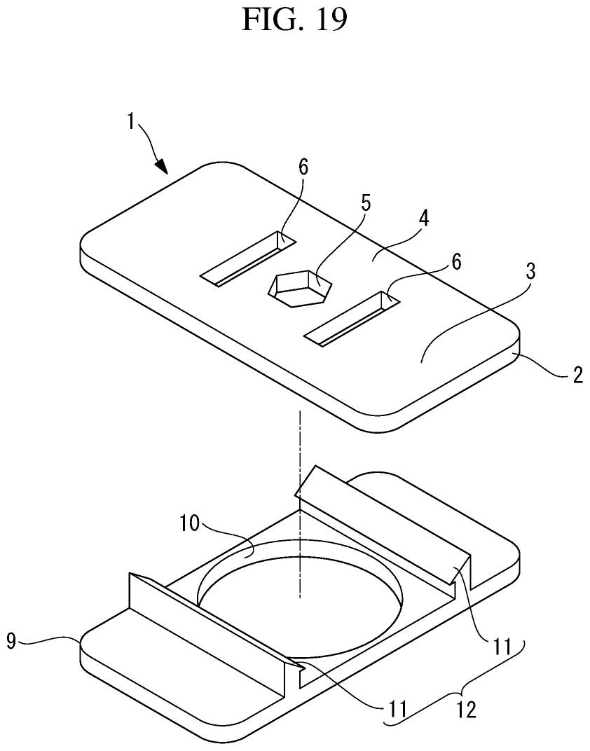

Furthermore, a positional-displacement prevention member 9 that prevents the ligament fixation device 1 fixed within the bone tunnel H from moving in the direction orthogonal to the axis of the bone tunnel H may be provided. As shown in FIG. 19, the positional-displacement prevention member 9 is a tabular member having a shape identical to the cross-sectional shape of the bone tunnel H and includes a through-hole 10 at the center for allowing the reconstruction ligament 200 to extend therethrough, and also includes an attachment section 11 for attaching the positional-displacement prevention member 9 to the fixation device body 2 of the ligament fixation device 1 in a stacked fashion in the thickness direction.

For example, the attachment section 11 is a pair of hooks 12 disposed on one surface of the positional-displacement prevention member 9 and extending parallel to each other while being separated from each other in the longitudinal direction by a distance equal to the width of the fixation device body 2 along the lateral axis Y. When the fixation device body 2 is disposed between the pair of hooks 12 and is pressed against the positional-displacement prevention member 9 in the thickness direction, the fixation device body 2 becomes fitted between the pair of hooks 12 and attached thereto. Accordingly, as shown in FIG. 20, the fixation device body 2 and the positional-displacement prevention member 9 are secured to each other in a stacked fashion in the thickness direction in a state where the longitudinal axis X of the fixation device body 2 and the lateral axis of the positional-displacement prevention member 9 are aligned with each other.

By securing the positional-displacement prevention member 9 to the fixation device body 2 fixed within the bone tunnel H, the positional-displacement prevention member 9 becomes hooked to the inner surface of the bone tunnel H, thereby preventing the fixation device body 2 from moving in the direction orthogonal to the axis of the bone tunnel H.

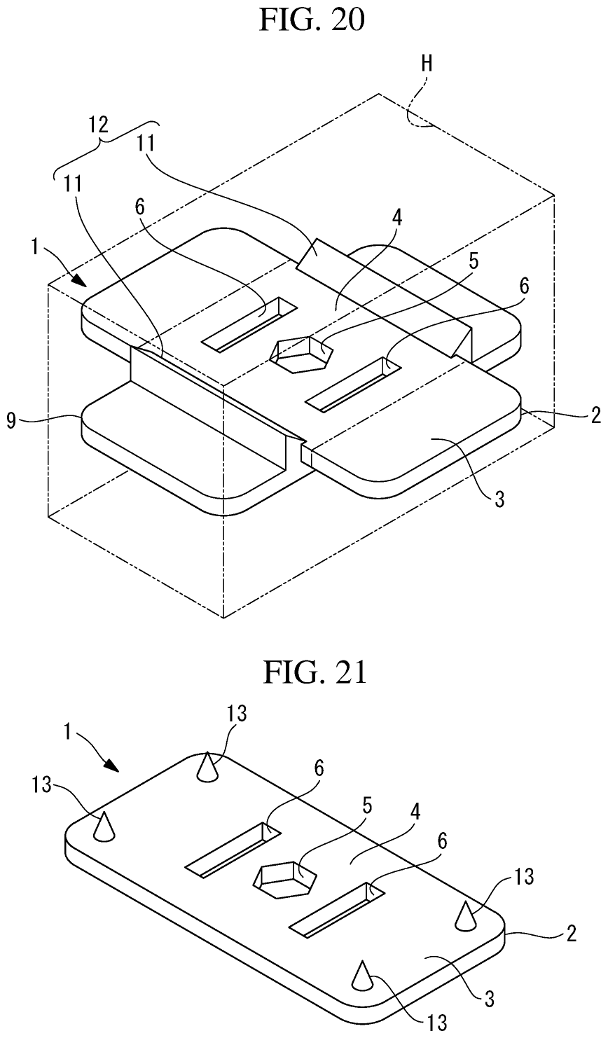

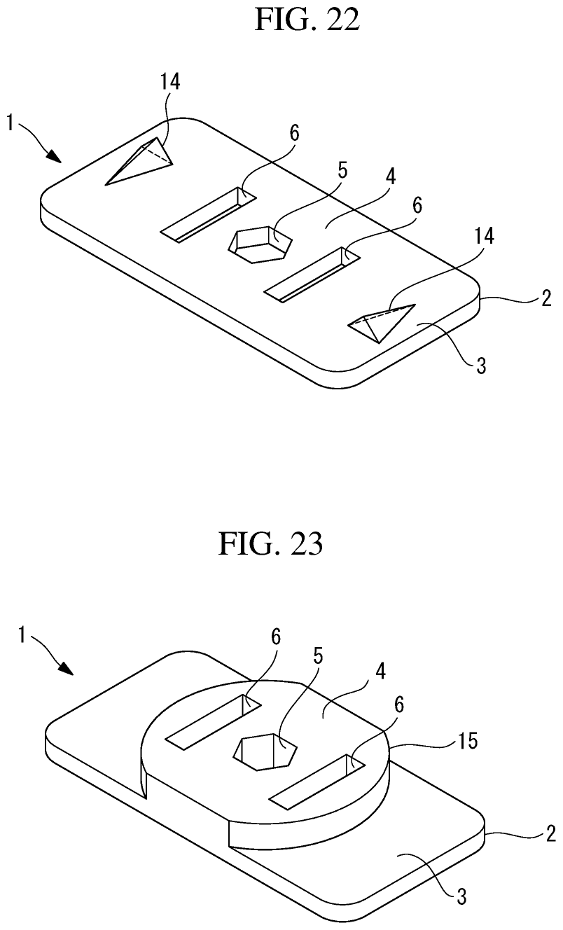

As an alternative to the use of the positional-displacement prevention member 9, the fixation device body 2 may be provided with a positional-displacement prevention section 13. For example, as shown in FIG. 21, the positional-displacement prevention section 13 may include protrusions that are provided on at least one of the front face and the rear face of the fixation device body 2 and that are to dig into the cancellous bone C. As another alternative, protrusions 14 having a shape hooking toward one of the rotational directions may be used as the positional-displacement prevention member 9, as shown in FIG. 22, such that each protrusion 14 facilitates the rotation in the rotational direction during installation but stops the rotation in the opposite direction.

Furthermore, as shown in FIG. 23, a cylindrical protrusion 15 having a diameter equal to the minimum inner dimension of the bone tunnel H may be provided at the center of the fixation device body 2 and near the second through-holes 6 serving as the mounting section to which the reconstruction ligament 200 is to be mounted. Accordingly, as shown in FIG. 24, in a state where the fixation device body 2 is fixed to the bone tunnel H, the fixation device body 2 can be prevented from moving in the direction orthogonal to the longitudinal axis L of the bone tunnel H.

The ligament graft serving as the reconstruction ligament 200 is directly inserted into the second through-holes 6 serving as the mounting section. Alternatively, a suture for hooking the ligament graft may be inserted and be formed into a loop. After the reconstruction ligament 200 is secured, the initial tension applied to the reconstruction ligament 200 may be adjusted by adjusting the length of the suture.

Although the second through-holes 6 are two slits provided at opposite sides of the first through-hole 5 in the direction of the longitudinal axis X, the configuration is not limited to this. The second through-holes 6 may simply be a plurality of through-holes provided at opposite sides of the first through-hole 5 in the direction of the longitudinal axis X.

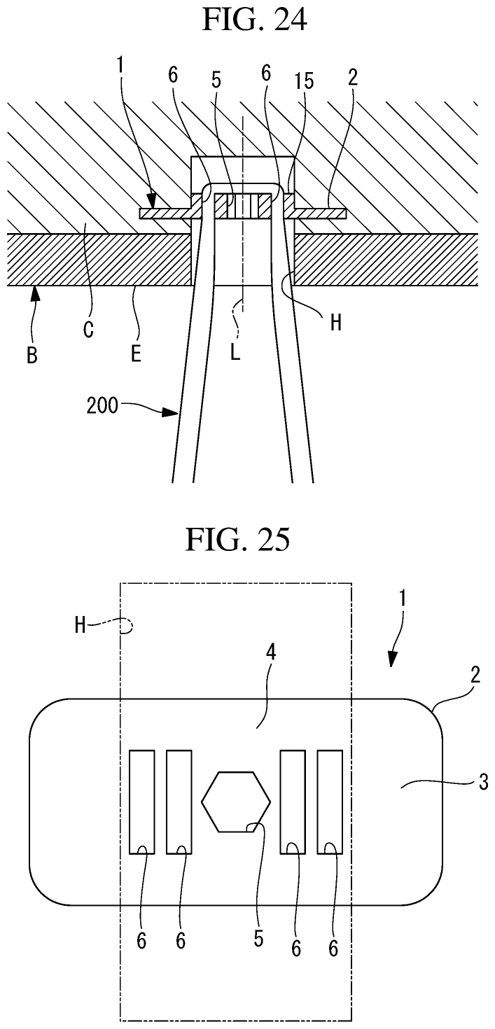

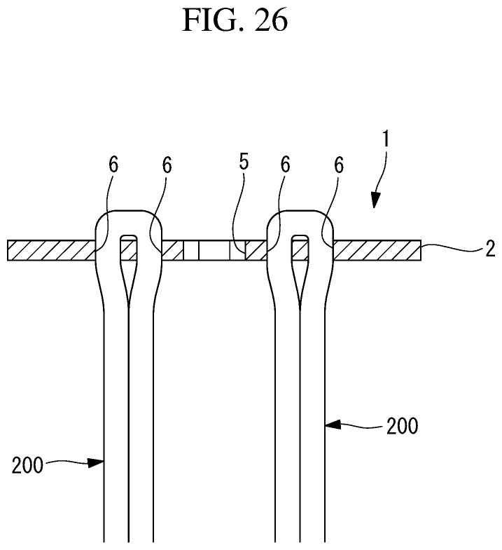

For example, if the second through-holes 6 are four slits provided at opposite sides of the first through-hole 5 in the direction of the longitudinal axis X, as shown in FIG. 25, a single reconstruction ligament 200 having a bundle of ligament grafts can be mounted, as shown in FIG. 26, so that the strength per ligament graft can be increased.

The above-described embodiment also leads to the following aspects.

An aspect of the present disclosure provides a ligament fixation method including forming a bone tunnel having a fixed noncircular cross section in a bone, mounting a ligament or a pulling member to a ligament fixation device having a maximum width smaller than a maximum length of the bone tunnel and larger than a minimum dimension of the bone tunnel, inserting the ligament fixation device into the bone tunnel, rotating the ligament fixation device around an axis of the bone tunnel, and attaching the ligament fixation device to the bone.

According to this aspect, the ligament fixation device having the ligament or the pulling member mounted thereto is inserted into the bone tunnel formed in the bone and having the fixed noncircular cross section. Then, after the ligament fixation device is inserted into the bone tunnel, the ligament fixation device is rotated around the axis of the bone tunnel. Accordingly, the section with the maximum width in the ligament fixation device can be disposed in the bone at the location at which the inner dimension of the bone tunnel is minimum, and the mounted ligament or pulling member can be disposed in a state where the ligament or the pulling member extends outward from the bone tunnel.

In this case, when the ligament fixation device is rotated within the bone tunnel, the bone in the area where the section with the maximum width in the ligament fixation device has moved is removed, so that a space is formed. However, because the ligament fixation device moves around the axis of the bone tunnel, a space is not formed in an area adjacent to the ligament fixation device in the longitudinal direction of the bone tunnel. Thus, even if the reconstructed ligament repeatedly receives tension acting in the axial direction of the bone tunnel, the ligament fixation device has no room to move in the axial direction of the bone tunnel, so that the ligament can be maintained in a stable fixed state.

Another aspect of the present disclosure provides a ligament fixation method including forming a bone tunnel having a fixed noncircular cross section from a surface of a bone, mounting a ligament or a pulling member to a ligament fixation device having a wide section with a width smaller than a maximum inner dimension of the cross section of the bone tunnel and larger than a minimum inner dimension of the cross section of the bone tunnel, inserting the ligament fixation device into the bone tunnel, rotating the ligament fixation device when the ligament fixation device is disposed within the bone tunnel, and setting the wide section at a location at which the inner dimension of the bone tunnel is minimum.

In the above aspect, the ligament fixation device may be inserted into the bone tunnel in a state where an axis of the ligament fixation device is aligned with an axis of the bone tunnel.

Furthermore, in the above aspect, the ligament fixation method may further include connecting a connector disposed at a distal end of a rod-like shaft to a torque applier of the ligament fixation device before inserting the ligament fixation device having the ligament or the pulling member mounted thereto into the bone tunnel, rotating the ligament fixation device within the bone tunnel by supplying torque around the axis of the bone tunnel to the shaft at a proximal end of the shaft, and disconnecting the connector from the torque applier and removing the shaft from the bone tunnel after the wide section is disposed at the location at which the inner dimension of the bone tunnel is minimum.

Furthermore, in the above aspect, the bone tunnel may be formed from the surface of the bone to a depth at which cancellous bone exists. Moreover, when the ligament fixation device is disposed within the cancellous bone, the ligament fixation device may be rotated around the axis, and the wide section may be set at the location at which the inner dimension of the bone tunnel is minimum.

Moreover, in the above aspect, the ligament fixation device may be tabular.

Another aspect of the present disclosure provides a ligament fixation device for fixing a ligament to a bone tunnel and including a fixation device body having a noncircular flat surface. The fixation device body has a mounting section for mounting the ligament or a pulling member thereto and a torque applier capable of applying torque around an axis orthogonal to the flat surface. The pulling member is provided for mounting the ligament.

According to this aspect, the fixation device body having the ligament or the pulling member mounted to the mounting section is inserted into the bone tunnel. Then, after the fixation device body is inserted into the bone tunnel, the torque applier rotates the fixation device body around the axis. Accordingly, the fixation device body is attached to the bone, and the mounted ligament or pulling member can be disposed in a state where the ligament or the pulling member extends outward from the bone tunnel.

In the above aspect, the ligament fixation device may be inserted into the bone tunnel in a state where an axis of the ligament fixation device is aligned with an axis of the bone tunnel.

According to this configuration, when the fixation device body is rotated within the bone tunnel, the bone in the area where the fixation device body has moved is removed, so that a space is formed. However, because the fixation device body moves in the direction orthogonal to the axis of the bone tunnel, a space is not formed in an area adjacent to the fixation device body in the longitudinal direction of the bone tunnel. Thus, even if the reconstructed ligament repeatedly receives tension acting in the axial direction of the bone tunnel, the fixation device body has no room to move in the axial direction of the bone tunnel, so that the ligament can be maintained in a stable fixed state.

Furthermore, in the above aspect, the fixation device body may be tabular.

According to this configuration, the fixation device body is rotated around the axis orthogonal to the surface thereof, so that the amount of the bone removed by the fixation device body is equivalent to the thickness of the fixation device body, whereby the ligament can be fixed with low invasiveness.

Furthermore, in the above aspect, the torque applier may be a through-hole having a noncircular cross-sectional shape and extending through the fixation device body in a thickness direction thereof.

According to this configuration, the jig inserted in the bone tunnel is engaged with the noncircular through-hole extending through the tabular fixation device body in the thickness direction, so that the jig can be supplied with torque around the axis orthogonal to the surface of the fixation device body.

Accordingly, the fixation device body disposed within the bone can be easily rotated around the axis outside the bone tunnel. The torque applier for applying torque to the tabular fixation device body can be easily realized by means of the through-hole extending through the fixation device body in the thickness direction.

Furthermore, in the above aspect, the torque applier may include a plurality of through-holes extending through the fixation device body in the thickness direction.

According to this configuration, the jig inserted in the bone tunnel is simultaneously engaged with the plurality of through-holes extending through the tabular fixation device body in the thickness direction, so that the jig can be supplied with torque around the axis orthogonal to the surface of the fixation device body.

Accordingly, the fixation device body disposed within the bone can be easily rotated around the axis outside the bone tunnel. The torque applier for applying torque to the tabular fixation device body can be easily realized by means of the through-holes extending through the fixation device body in the thickness direction.

Furthermore, in the above aspect, the mounting section may be a through-hole that extends through the fixation device body in the thickness direction and through which the ligament or the pulling member is extendable.

According to this configuration, the ligament or the pulling member can be easily mounted to the fixation device by simply inserting the ligament or the pulling member through the through-hole provided in the fixation device.

Furthermore, in the above aspect, at least a part of an edge of the fixation device body may be provided with a sharp edge that decreases in thickness toward a tip.

According to this configuration, when the fixation device body is to be rotated within the bone, the sharp edge is disposed as the leading edge in the rotational direction, so that the fixation device body can easily cut into the bone by using the sharp edge as a blade, thereby facilitating the installation process of the fixation device body.

Another aspect of the present disclosure provides a ligament fixation system including the aforementioned ligament fixation device and an installation jig to be used when the ligament fixation device is to be installed in a bone. The ligament fixation device includes a wide section extending along a first axis and a narrow section having a width smaller than that of the wide section and extending along a second axis intersecting the first axis. The installation jig includes a rod-like shaft having a cross-sectional shape with an outer diameter smaller than the width of the narrow section, a connector provided at a distal end of the shaft and detachably connected to the torque applier along the axis in a state where the axis is aligned with a longitudinal axis of the shaft, and a torque supplier that is provided at a proximal end of the shaft and that supplies torque around the longitudinal axis to the shaft.

According to this aspect, the connector provided at the distal end of the shaft of the installation jig is connected to the torque applier of the ligament fixation device equipped with the ligament or the pulling member, and the ligament fixation device can be inserted into the bone tunnel. Then, at the location where the ligament fixation device has reached the bone, the torque supplier disposed outside the bone supplies the shaft with torque around the longitudinal axis, so that the ligament fixation device can be rotated around the axis of the bone tunnel within the bone. After the ligament fixation device is rotated, the connector is disconnected from the torque applier, and the shaft is removed outside the bone tunnel, whereby the ligament fixation device can be kept inside the bone in a fixed state.

In the above aspect, the installation jig may include a step on which the surface of the fixation device body abuts when the ligament fixation device is installed in the bone.

1. A ligament fixation device for fixing a ligament to a bone tunnel, the ligament fixation device comprising:
a fixation device body having a flat surface, the fixation device body including:
a mounting section configured to mount a pulling member, the pulling member being configured to mount the ligament;
a torque application portion configured to apply torque about an axis orthogonal to the flat surface; and
a pair of through-holes spaced from the torque application portion on the flat surface.

2. The ligament fixation device according to claim 1,
wherein the pair of through-holes are disposed in parallel to each other.

3. The ligament fixation device according to claim 1,
wherein the fixation device body is tabular.

4. The ligament fixation device according to claim 3,
wherein the torque application portion includes a through-hole that has a noncircular cross-sectional shape and extends through a thickness direction of the fixation device body.

5. The ligament fixation device according to claim 4,
wherein the torque application portion includes a plurality of through-holes extending through the fixation device body in the thickness direction.

6. The ligament fixation device according to claim 4,
wherein a portion an edge of the fixation device body is provided with a sharp edge that decreases in thickness toward a tip.

7. A ligament fixation system comprising:
the ligament fixation device according to claim 1; and
an installation jig configured to be used when the ligament fixation device is installed in a bone, wherein:
the ligament fixation device includes a wide section extending along a first axis and a narrow section having a width smaller than that of the wide section and extending along a second axis intersecting the first axis, and
the installation jig includes:
a rod-like shaft having a cross-sectional shape with an outer diameter smaller than the width of the narrow section,
a connector provided at a distal end of the shaft and detachably connected to the torque application portion along the axis when the axis is aligned with a longitudinal axis of the shaft, and
a torque supplying portion that is provided at a proximal end of the shaft and that supplies torque around the longitudinal axis to the shaft.

8. The ligament fixation system according to claim 7,
wherein the installation jig includes a step configured to abut the flat surface of the fixation device body when the ligament fixation device is installed in the bone.
